(19) United States
(12) Patent Application Publication
(10) Pub. No.: US 2025/0367021 A1
(43) Pub. Date: Dec. 4, 2025

(54) PALATAL IMPLANT AND RELATED METHODS

(71) Applicant: Serene Sleep, Inc., Wilmington, DE (US)

(72) Inventors: Lawrence Bright, San Francisco, CA (US); Kelsey Seto, San Francisco, CA (US)

(73) Assignee: Serene Sleep, Inc., Wilmington, DE (US)

(21) Appl. No.: 19/219,707

(22) Filed: May 27, 2025

Related U.S. Application Data
(60) Provisional application No. 63/652,597, filed on May 28, 2024.
(60) Provisional application No. 63/697,082, filed on Sep. 20, 2024.

(51) Int. Cl. A61F 5/56; A61L 27/18; D02G 3/02; D02G 3/34; D02G 3/44; D04C 1/02; D04C 1/06

(52) U.S. Cl. CPC A61F 5/566; A61L 27/18; D02G 3/02; D02G 3/34; D02G 3/448; D04C 1/02; D04C 1/06; D10B 2331/04; D10B 2509/00

(57) ABSTRACT

Implementations of palatal implants may include a core having a plurality of air jet textured core yarns, eight triaxial yarns coupled around the core and parallel to the core, wherein each of the eight triaxial yarns are air jet textured and have a plurality of looped filaments, 24 biaxial yarns braided around the core in a one over one under one pattern, a first weld offset from a first end of the palatal implant by 0.8 millimeters to 1.2 millimeters, and a second weld offset from a second end of the palatal implant by 0.8 millimeters to 1.2 millimeters. The looped filaments in the triaxial yarns may protrude through openings between the biaxial yarns, the palatal implant may have a total braid weight of 7,000 denier to 12,500 denier, and the palatal implant may be configured to stiffen a palate through tissue ingrowth into the palatal implant.

### CROSS REFERENCE TO RELATED APPLICATIONS

This document claims the benefit of the filing date of U.S. Provisional Patent Application 63/652,597, entitled “Fibrous Braided Implant for Treatment of Snoring and Sleep Apnea” to Serene Sleep, Inc. which was filed on May 28, 2024, and U.S. Provisional Patent Application 63/697,082, entitled “Fibrous Braided Implant for Treatment of Snoring and OSA via Fibrotic Response” to Serene Sleep, Inc. which was filed on Sep. 20, 2024, the disclosures of each of which are hereby incorporated entirely herein by reference.

### BACKGROUND

### 1. Technical Field

Aspects of this document relate generally to medical implant devices. More specific implementations involve palatal implants for the soft palate.

### 2. Background

The sound of snoring is a vibratory response to air passing over the soft palate in the back of the mouth. When air passes across the relaxed soft palate, it flutters, often audibly. In many cases, the soft palate relaxes so much that it collapses onto the back of the throat, restricting flow into the airway and resulting in obstructive sleep apnea.

### SUMMARY

Implementations of palatal implants may include a core having a plurality of air jet textured core yarns and eight triaxial yarns coupled around the core and parallel to the core. Each of the eight triaxial yarns may be air jet textured and have a plurality of looped filaments. Implementations of palatal implants may also include 24 biaxial yarns braided around the core in a one over one under one pattern, a first weld offset from a first end of the palatal implant by 0.8 millimeters to 1.2 millimeters, and a second weld offset from a second end of the palatal implant by 0.8 millimeters to 1.2 millimeters. The looped filaments in the triaxial yarns may protrude through openings between the biaxial yarns. The triaxial yarns may each have a weight between 250-600 denier. The biaxial yarns may each have a weight between 50-100 denier. The palatal implant may have a flexural modulus between 1.0 MPa and 3.0 MPa. The palatal implant may have a total braid weight of 7,000 denier to 12,500 denier. The palatal implant may be configured to stiffen a palate through tissue ingrowth into the palatal implant.

Implementations of palatal implants may include one, all, or any of the following:

The core yarns, biaxial yarns, and triaxial yarns may be polyethylene terephthalate (PET).

The palatal implant may have a length of less than 20 millimeters.

The palatal implant may have a diameter between one millimeter and two millimeters.

The palatal implant may have a linear density of 0.78 grams per meter (g/m) to 1.39 grams per meter (g/m).

The palatal implant may have a solidity of 50%.

The 24 biaxial yarns may be flat drawn yarns.

The palatal implant may include a plurality of air jet textured core yarns that are 400 denier and have 108 filaments per yarn, eight triaxial yarns that are 400 denier and have 108 filaments per yarn, and 24 biaxial yarns that are 70 denier and have 30 filaments per yarn.

Implementations of a method of constructing a palatal implant may include air jet texturing a plurality of core yarns, air jet texturing eight triaxial yarns, loading and evenly spacing the eight triaxial yarns on eight carriers of a 48 carrier braiding machine, loading 24 biaxial yarns on 24 carriers of the 48 carrier braiding machine, orienting each of the 24 biaxial yarns between 40 degrees and 60 degrees or negative 40 degrees and negative 60 degrees, braiding the 24 biaxial yarns in a one under one over one configuration to form a braided structure, forming a plurality of welds along the braided structure, and cutting the braided structure to form a plurality of palatal implants. The 48 carrier braiding machine may be loaded in a half-load configuration when the eight triaxial yarns and the 24 biaxial yarns are loaded onto the 48 carrier braiding machine.

Implementations of methods of constructing a palatal implant may include one, all, or any of the following:

The air jet texturing of the core yarns and the air jet texturing of the triaxial yarns may have less than 50% of the input yarn as core yarn and greater than 50% of the input yarn as effect yarn.

The braided structure of the palatal implant may have a total weight of 7,000 denier to 12,500 denier.

The mean height of a surface of the texture of the palatal implant may be greater than 50 micrometers.

The combined overfeed of the input yarn may be greater than 20% across both the core yarn and the effect yarn.

Each of the plurality of air jet textured yarns may be 400 denier and include 108 filaments per yarn, each of the eight triaxial yarns may be 400 denier and may have 108 filaments per yarn, and each of the 24 biaxial yarns may be 70 denier and contain 30 filaments per yarn.

Implementations of a method of constructing a palatal implant may include air jet texturing a plurality of core yarns, air jet texturing eight triaxial yarns, loading and evenly spacing the eight triaxial yarns on eight carriers of a 48 carrier braiding machine, loading 24 biaxial yarns on 24 carriers of the 48 carrier braiding machine, orienting each of the 24 biaxial yarns between 40 degrees and 60 degrees or between negative 40 degrees and negative 60 degrees, braiding the 24 biaxial yarns in a one under one over one configuration to form a braided structure, forming a plurality of welds along the braided structure and cutting the braided structure to form a plurality of palatal implants. The 48 carrier braiding machine may be loaded in a half-load configuration when the eight triaxial yarns and the 24 biaxial yarns are loaded onto the 48 carrier braiding machine. The triaxial yarns of the plurality of palatal implants may have looped filaments that protrude through openings between the biaxial yarns. The triaxial yarns of the plurality of palatal implants may each have a weight of between 250-600 denier. The biaxial yarns of the plurality of palatal implants may each have a weight of between 50-100 denier. The palatal implant may have a flexural modulus between 1.0 MPa and 3.0 MPa. The palatal implant may have a total weight of 7,000 denier to 12,500 denier. The palatal implant may be configured to stiffen a palate via its inherent added stiffness and support and/or via tissue ingrowth into and other tissue growth around and encapsulating the palatal implant.

Implementations of methods of constructing a palatal implant may include one, all, or any of the following:

The plurality of palatal implants may have a pick count of 40 picks per inch (ppi) to 50 picks per inch (ppi).

The air jet texturing of the core yarns and the air jet texturing of the triaxial yarns may have less than 50% of the input yarn as core yarn and greater than 50% of the input yarn as effect yarn.

The braided structure of the palatal implant may have a linear density of 0.78 grams per meter (g/m) to 1.39 grams per meter (g/m).

Each palatal implant of the plurality of palatal implants may have a first weld between 0.8-1.2 millimeters from a first end and may have a second weld between 0.8-1.2 millimeters from a second end.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

Implementations will hereinafter be described in conjunction with the appended drawings, where like designations denote like elements, and:

FIG. 1 is a perspective view of a palatal implant;

FIG. 2 is a top view of a palatal implant;

FIG. 3 is a side view of a palatal implant;

FIG. 4 is a view of a braided structure;

FIG. 5 is a zoomed in view of a braided structure;

FIG. 6 is a view of a braiding structure including looped filaments;

FIG. 7 is a view of 48 carrier braiding machine; and

FIG. 8 is a view of a loaded braiding machine.

### DESCRIPTION

This disclosure, its aspects and implementations, are not limited to the specific components, assembly procedures or method elements disclosed herein. Many additional components, assembly procedures and/or method elements known in the art consistent with the intended palatal implant will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such palatal implants, and implementing components and methods, consistent with the intended operation and methods.

As used herein, terms of direction and orientation, such as, by non-limiting example, top, bottom, high, low, up, down, first end, and second end are understood in relation to the orientation of the palatal implant when the length of the palatal implant is positioned horizontally or parallel to the ground. As used herein, terms of coupling, such as, by non-limiting example, fixedly, directly, and adjustably, are understood to include a variety of coupling methods, such as, by non-limiting example, welded, interlocking, braiding, or any other adhesive or mechanical method of attachment.

As used herein, identification terms such as, by non-limiting example, core, triaxial, and biaxial are understood to identify parts and materials of the palatal implant and also parts and materials that are to be used to form the palatal implant. For example, yarns that will be used to form a core but have not yet been assembled to form a core, may still be referred to as core yarns. As used herein, braiding machine terminology, methods, and components should be given their plain and ordinary meanings that are known in the art and should be apparent to one of ordinary skill in the art.

The sound of snoring may be a vibratory response to air passing over the soft palate in the back of the mouth. The soft palate may control airflow through the nose and mouth. During sleep, the soft palate may relax. When air passes across the relaxed soft palate, it may cause the soft palate to audibly flutter. This dynamic response may result in snoring. In some cases, the soft palate may relax and collapse onto the back of the throat. This collapse may restrict airflow and result in obstructive sleep apnea (OSA).

Implementations of the palatal implants disclosed herein may stiffen the soft palate to prevent the soft palate from collapsing on the back of the throat. This stiffening may correspondingly alter the dynamic response that results in snoring and prevent or lessen the soft palate collapse that can cause sleep apnea. The palatal implants disclosed herein may stiffen the soft palate through several methods. First, the palatal implants may directly stiffen the soft palate. The structure of the palatal implants itself may directly stiffen the tissue when it is implanted by supporting the soft palate region. Next, the palatal implants disclosed herein may stiffen the soft palate through fibrotic stiffening. The palatal implants material may prompt a fibrotic response in the tissue, and scar tissue may develop to encapsulate the palatal implant. The scar tissue may stiffen the tissue and may form a bridge between the palatal implant and the hard palate, creating a broader stiffening zone. Finally, the palatal implants disclosed herein may stiffen the soft palate through composite reinforcement. The scar tissue may grow around the palatal implants and throughout the palatal implants. This growth may create a binding matrix which may allow the fibers to pass shear loads through each other as opposed to slip past each other. In turn, the binding matrix may further stiffen the palatal implant disclosed herein.

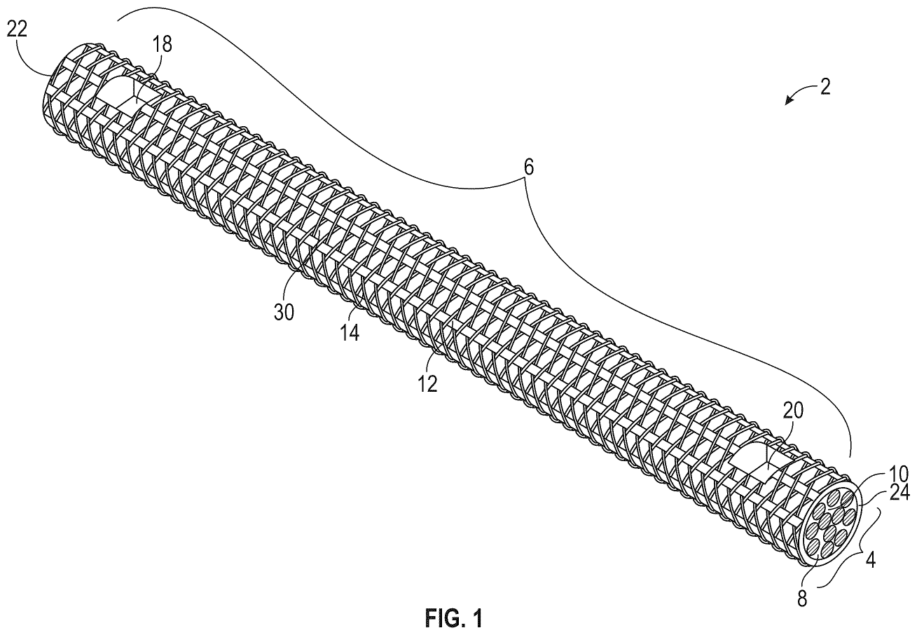

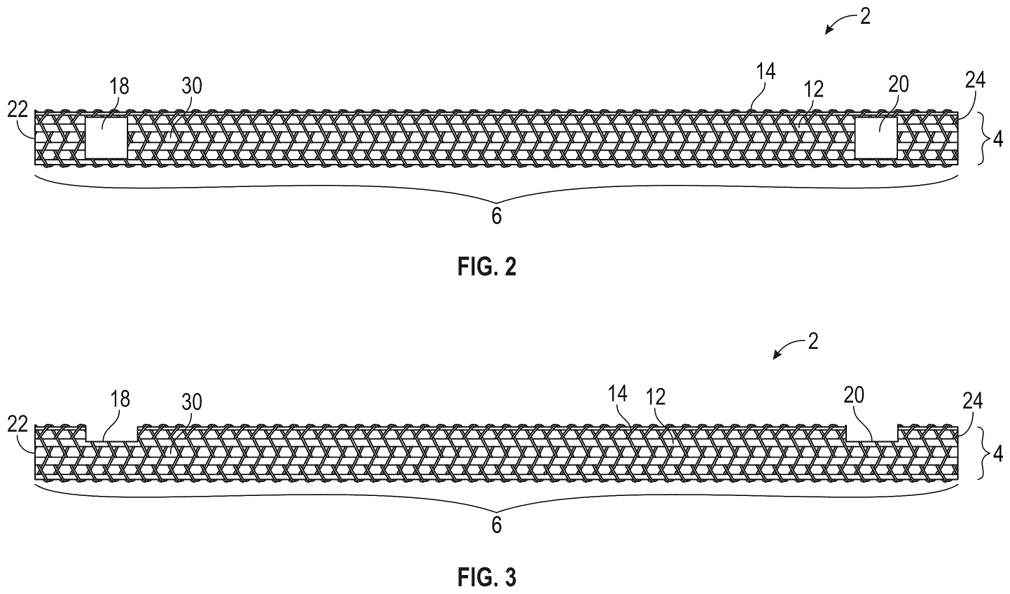

Referring to FIGS. 1-3 a palatal implant 2 is illustrated. The palatal implant 2 may have a diameter 4 between one millimeter and two millimeters and may have a length 6 that is less than 20 millimeters. The palatal implant 2 may be small enough to fit into a standard 14RW gauge hypodermic needle. The palatal implant 2 may have a total denier weight of 10,200 denier. In other implementations, the palatal implant 2 may have a total denier weight of 7,000 denier to 14,700 denier.

The palatal implant 2 may be cylindrical and have a circular cross-sectional shape. In other implementations, the palatal implant 2 may have, by non-limiting example, a triangular, rectangular, pentagonal, hexagonal, heptagonal, or octagonal cross-sectional shape. The palatal implant 2 has core yarns 8 that make up the center or core 10 of the cylinder. The palatal implant 2 has triaxial yarns 12 and biaxial yarns 14 that are braided together and make up the perimeter of the cylindrical shape of the palatal implant 2. The triaxial yarns 12 and the biaxial yarns 14 may be braided around and coupled around the core 10 of the palatal implant 2.

In various implementations, the palatal implant 2 may be made of polyethylene terephthalate (PET). In other implementations, the palatal implant 2 may be made of, by non-limiting example, polypropylene, polymethyl methacrylate, expanded polytetrafluroethylene (ePTFE) or any other biocompatible polymer. PET is a polymer which can, under particular conditions, promote a fibrotic response in human tissue that results in the formation of scar tissue. The palatal implant 2 may be made with a material that is biocompatible and non-bioabsorbable. In other implementations, the palatal implant 2 may be made with bio-absorbable material or a combination of bio-absorbable and non-bioabsorbable materials. The material of the palatal implant 2 may be compatible with ethylene oxide sterilization. The material of the palatal implant 2 may hold an acceptable level of ethylene oxide residuals. In other implementations, the palatal implant 2 may not hold a level of ethylene oxide residuals above acceptable levels specified by ISO 11135 and ISO 10993-7. The material of the palatal implant 2 may be capable of remaining chemically stable and structurally unchanged for at least two years when stored in typical storage conditions. The palatal implant 2 may have a total braid weight of 7,000 denier to 12,500 denier. In particular implementations, the palatal implant 2 total braid weight may be 10,200 denier. The palatal implant 2 may have a linear density of 0.78 grams per meter (g/m) to 1.39 grams per meter (g/m). In particular implementations, the palatal implant 2 may have a linear density of 1.13 grams per meter (g/m).

Still referring to FIGS. 1-3, the palatal implant 2 may have a diameter 4 of between one to two millimeters. In particular implementations, the palatal implant may have a diameter of 1.5 millimeters. In various implementations, the palatal implant 2 may have a length thatis 18 millimeters. In other implementations, the palatal implant 2 may have a length that is less than or more than 20 millimeters.

The palatal implant 2 may have a mass of 20 milligrams. In other implementations, the palatal implant 2 may have a mass of less than 20 milligrams or more than 20 milligrams.

The palatal implant 2 may have a linear density of 1.1 grams per meter (g/m). In other implementations, the palatal implant 2 may have a linear density between 0.78 grams per meter (g/m) and 1.40 grams per meter (g/m).

The palatal implant 2 may have a bounding volume of 31.9 cubic millimeters (mm3). In other implementations, the palatal implant 2 may have a bounding volume that is less than 31.9 cubic millimeters (mm3) or more than 31.9 cubic millimeters (mm3).

The palatal implant 2 may have a density of 0.63 grams per cubic centimeter (g/cm³). In other implementations, the palatal implant 2 may have a density less than 0.69grams per cubic centimeter (g/cm³) or more than 0.63 grams per cubic centimeter (g/cm³).

The palatal implant 2 may have a solidity of 45%. In other implementations, the palatal implant 2 may have a solidity that is between 30% and 70%.

The palatal implant 2 may have a flexural modulus of 1.51 megapascals (MPa). In other implementations, the palatal implant 2 may have a flexural modulus that is between 1.0 megapascal (MPa) and 3.0 megapascals (MPa).

The palatal implant 2 may have a surface roughness (arithmetical mean height of the surface) that is greater than 50 micrometers (μm). In other implementations, the palatal implant 2 may have a surface roughness (arithmetical mean height of surface) that is equal to 50 micrometers (μm) or less than 50 micrometers (μm). The palatal implant 2 may have a maximum amplitude between the peak and valley of the surface texture that is greater than 300 micrometers (μm). In other implementations, the palatal implant 2 may have a maximum amplitude between the peak and valley of the surface texture that is equal to 300 micrometers (μm) or less than 300 micrometers (μm). The maximum amplitude between the peak and valley of the surface texture may also be referred to as the sum of the largest peak height value and the largest pit depth value. The standard deviation of the surface height of the palatal implant 2 may be more than 40 micrometers (μm). In other implementations, the standard deviation of the surface height of the palatal implant 2 may be equal to 40 micrometers (μm) or less than 40 micrometers (μm). The standard deviation of the surface height of the palatal implant 2 may also be referred to as the root mean square value of ordinate values of the surface. The surface texture of the palatal implant promotes scar tissue growth around and within the palatal implant when placed within a soft palate.

The palatal implant 2 may include a first weld 18 and a second weld 20. The first weld 18 and the second weld 20 on the palatal implant 2 may be cubic and may have dimensions of one millimeter by one millimeter by one millimeter. In other implementations, each dimension of the first weld 18 and the second weld 20 on the palatal implant 2 may be as small as 0.9 millimeters, as large as 1.1 millimeters, or may be any length between 0.9 millimeters and 1.1 millimeters. The first weld 18 may be one millimeter from a first end 22 of the palatal implant 2. The second weld 20 may be one millimeter from a second end 24 of the palatal implant 2. In other implementations, the first weld 18 may be between 0.8 millimeters and 1.2 millimeters from the first end 22 of the palatal implant 2 and the second weld 20 may be between 0.8 millimeters and 1.2 millimeters from the second end 24 of the palatal implant 2.

Referring to FIG. 1, the palatal implant 2 may include a core 10 made up of core yarns 8. The core yarns 8 may be positioned on the interior of the palatal implant 2. Referring to FIG. 1, the core 10 and core yarns 8 are illustrated. In particular implementations, the palatal implant 2 may include a quantity of eleven core yarns 8. In other implementations, the palatal implant 2 may include less than eleven core yarns 8 or more than eleven core yarns 8. In other implementations, the core may be comprised of a bundle of similar denier weight as a single yarn. The core yarns 8 may be made of polyethylene terephthalate (PET) material. In other implementations, the core yarns 8 may be made of, by non-limiting example, polypropylene, polymethyl methacrylate, any material disclosed herein, or any other biocompatible polymer. In particular implementations, each core yarn 8 may include one hundred and eight air jet textured filaments. In other implementations, the core yarns 8 may include less than one hundred and eight air jet textured filaments or more than one hundred and eight air jet textured filaments. Each core yarn 8 may have a weight of 250 to 600 denier. In particular implementations, each core yarn 8 may have a weight of 400 denier. In other implementations, each core yarn 8 may have a weight between 250 denier and 400 denier or a weight between 400 denier and 600 denier. The resulting core 10 of the palatal implant 2, made up of core yarns 8, will be highly fibrous and will result in a palatal implant 2 of low solidity. The voids produced by the fiber loops inside the core 10 of the palatal implant 2 may promote and/or allow scar tissue growth within the interior of the palatal implant 2 and ultimately increase palatal stiffness. The core yarns 8 may be axially oriented (also known as horizontally oriented or oriented at zero degrees). In other implementations, the core yarns 8 may be oriented at substantially zero degrees. In particular implementations, the core yarns 8 may be axially oriented within the core 10 of the palatal implant 2. In other implementations, the core yarns 8 may be generally axially oriented and spiraled or woven around each other within the core 10 of the palatal implant 2. The axially oriented core yarns 8 may provide axial stiffness to the palatal implant 2 in the tensile direction. The axially oriented core yarns 8 may also provide bending and compressive stiffness to the palatal implant 2.

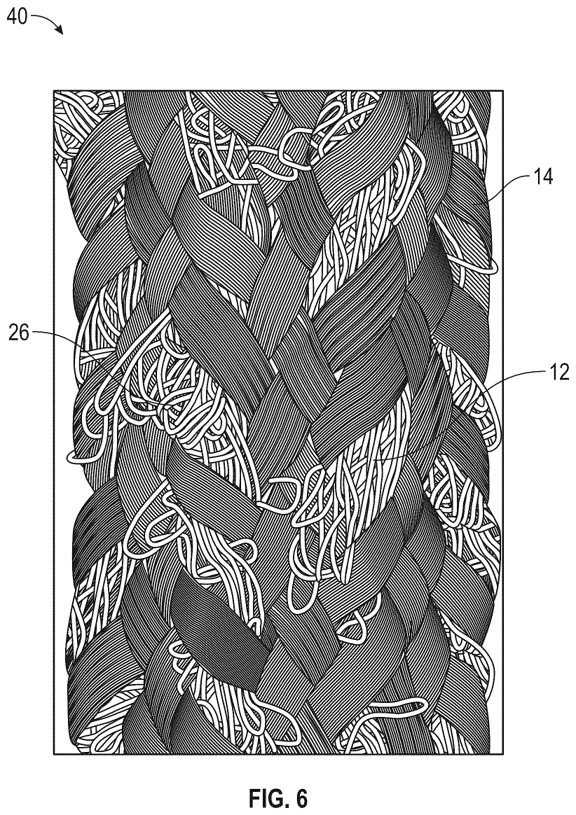

Referring to FIGS. 1-6, the palatal implant 2 may include triaxial yarns 12. In particular implementations, the palatal implant 2 may include eight triaxial yarns. In other implementations, the palatal implant may include less than eight triaxial yarns or more than eight triaxial yarns 12. The palatal implant 2 may be made of polyethylene terephthalate (PET) material. In other implementations, the core yarns 8 may be made of, by non-limiting example, polypropylene, polymethyl methacrylate, any material disclosed herein, or any other biocompatible polymer. Each triaxial yarn 12 may be air jet textured. In particular implementations, each triaxial yarn 12 may include one hundred and eight filaments. In other implementations, each triaxial yarn 12 may include less than one hundred and eight filaments or more than one hundred and eight filaments. Each triaxial yarn 12 may have a weight of 250to 600 denier. In particular implementations, each triaxial yarn 12 may have a weight of 400 denier. In other implementations, each triaxial yarn 12 may have a weight between 250 denier and 400 denier or a weight between 400 denier and 600 denier. The resulting triaxial yarns 12 of the palatal implant 2 will be highly fibrous and will result in a palatal implant 2 of low solidity. The voids produced by the looped filaments 26 of the triaxial yarns 12 may promote and/or allow scar tissue growth on the exterior surface and within the exterior of the palatal implant 2 and ultimately increase palatal stiffness. The triaxial yarns 12 may be axially oriented. In other implementations, the triaxial yarns 12 may be substantially axially oriented. The axially oriented triaxial yarns 12 may provide axial stiffness to the palatal implant 2 in the tensile direction. The axially oriented triaxial yarns 12 may also provide bending stiffness to the palatal implant 2. The triaxial yarns 12 may be braided with the biaxial yarns 14. The triaxial yarns 12 may be braided around and coupled around the exterior perimeter of the core yarns 8.

Referring to FIGS. 1-6, the palatal implant 2 may include biaxial yarns 14. Referring to FIGS. 1-6, biaxial yarns 14 are illustrated. The biaxial yarns 14 may be made of high tenacity polyethylene terephthalate (HTPET) material. In other implementations, the biaxial yarns 14 may be made from, by non-limiting example, polypropylene, polymethyl methacrylate, or any other biocompatible polymer. In particular implementations, each biaxial yarn 14 may include straight drawn (also known as flat drawn) filaments. In particular implementations, each biaxial yarn 14 may include thirty filaments. In other implementations, each biaxial yarn 14 may include less than thirty filaments or more than thirty filaments. Each biaxial yarn 14 may weigh 50 denier to 100 denier. In particular implementations, each biaxial yarn 14 may weigh 70 denier. In other implementations, each biaxial yarn 14 may weigh between 50 and 70 denier or between 70 and 100 denier. The biaxial yarns 14 may be oriented at approximately 45 degrees or negative 45 degrees relative to an axis perpendicular to a long length of the palatal implant. In other implementations, the biaxial yarns 14 may be oriented between 42.5 degrees and 47.5 degrees or between negative 42.5 and negative 47.5 degrees. In other implementations, the biaxial yarns 14 may be oriented between 40 degrees and 60degrees or between negative 40 degrees and negative 60 degrees. The size, weight, type, angle, and tension of the biaxial yarns result in openings through which the loops of the triaxial yarns may extend to promote the fibrotic response. The biaxial yarns 14 may provide the palatal implant 2 with axial stiffness in the tensile direction. The biaxial yarns 14 may also provide the palatal implant 2 with torsional stiffness and crosswise (90 degrees) bending stiffness. The biaxial yarns 14 may be braided with the triaxial yarns 12. The biaxial yarns 14 may be braided around and coupled around the exterior of the core 10 and core yarns 8.

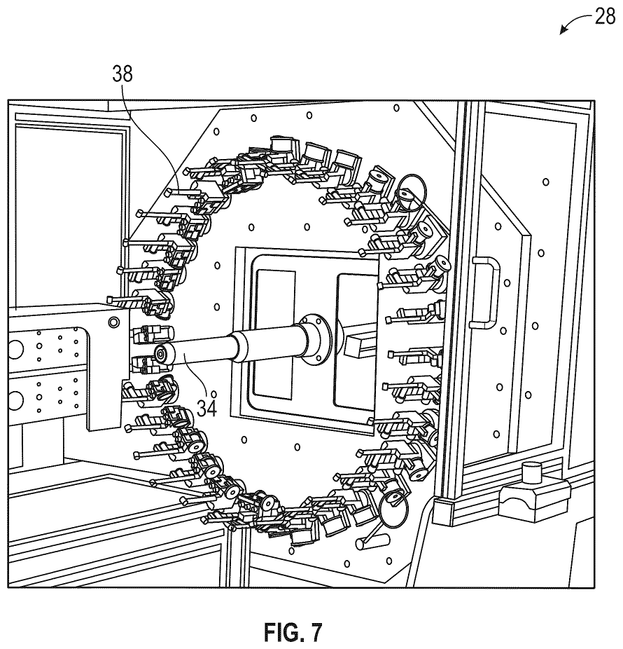

Referring to FIG. 7, the core yarns 8 and the triaxial yarns 12 may have looped filaments 26. Referring to FIG. 7, the looped filaments 26 of the triaxial yarns 12 are illustrated. These looped filaments 26 on the core yarns 8 and looped filaments 26 on the triaxial yarns 12 may be formed through air jet texturing the individual yarns before they are loaded onto a forty-eight carrier braiding machine 28. The triaxial yarns 12 may have looped filaments 26 that protrude through the openings 30 between the biaxial yarns 14 when the triaxial yarns 12 and biaxial yarns 14 are braided together. The looped filaments of the core yarns 8 and the protruding looped filaments 26 of the triaxial yarns 12 may be configured to promote and/or allow scar tissue growth after the palatal implant 2 has been implanted in a soft palate. The looped filaments 26 of the triaxial yarns 12 may be particularly effective at forming scar tissue because they may protrude through the openings 30 in the biaxial yarns 14 and therefore may be directly exposed to the exterior of the palatal implant 2. In turn, the exterior of the palatal implant 2 may be in direct contact with the interior tissue of the soft palate after implantation and may be very effective in promoting a fibrotic response of the soft palate tissue.

Referring to FIGS. 1-3, the palatal implant may have a first weld 18 and a second weld 20. Referring to FIGS. 1-3, a first weld 18 and a second weld 20 are illustrated. In other implementations, the palatal implant 2 may have one weld or more than two welds. The first weld 18 and the second weld 20 may be ultrasonic welds. In other implementations, the first weld 18 and the second weld 20 may be, by non-limiting example, horn welds, spot welds, continuous drive welds, or any other type of sufficiently secure weld. The first weld 18 and the second weld 20 may be square shaped welds that are one millimeter by one millimeter. In other implementations, the first weld 18 and the second weld 20 may be, by non-limiting example, circular, triangular, pentagonal, hexagonal, heptagonal, or octagonal in shape. In other implementations, the first weld 18 and the second weld 20 may be cubic and may have a width of one millimeter, a length of one millimeter, and a depth of one millimeter. The first weld 18 may be located between 0.8 millimeters and 1.2 millimeters from a first end 22 of the palatal implant 2. The second weld 20 may be located between 0.8 millimeters and 1.2 millimeters from a second end 24 of the palatal implant 2. The first weld 18 and the second weld 20 may fuse the implant fibers together and may prevent unraveling. However, the location of the first weld 18 may result in fraying throughout the approximately 0.8 millimeters to 1.2 millimeters at the first end 22 of the palatal implant 2. The location of the second weld 20 may result in fraying throughout the approximately 0.8 millimeters to 1.2 millimeters at the second end 24 of the palatal implant 2. This fraying (or splaying) in the fibers at the first end 22 of the palatal implant 2 and the fraying of fibers at the second end 24 of the palatal implant 2 may promote fibrotic encapsulation at both the first end 22 of the palatal implant 2 and at the second end 24 of the palatal implant 2.

A method of forming a palatal implant 2 may include air jet texturing the core yarns 8. The core yarns 8 may be air jet textured before they are loaded onto the braiding machine 28. When air jet texturing the core yarns 8, less than 50% of the input yarn may be core yarns and greater than 50% of the input yarns may be effect yarn. In other implementations, greater than 50% of the input yarn may be core yarns and less than 50% of the input yarns may be effect yarn, or 50% of the input yarns may be core yarns and 50% of the input yarns may be effect yarn. When air jet texturing the core yarns 8, the combined over feed of the input yarn may be greater than 20% across both the core yarn and the effect yarn. In other implementations, the combined overfeed of the input yarn may be less than 20% across both the core yarn and the effect yarn or equal to 20% across both the core yarn and the effect yarn. In particular implementations, there may be a quantity of one, 100 denier, thirty-six filament core yarn fed into the air jet texturing process at 20% overfeed for every quantity of two, 100 denier, thirty-six filament effect yarn fed into the air jet texturing process at 40% overfeed. The resulting air jet textured core yarns 8 may be a fluffy or hairy and low-density fill of yarns. Because the core 10 of the palatal implant 2 will be filled with loopy air textured yarns, the core 10 may be highly fibrous and the palatal implant 2 may have a low solidity. The fluffy core yarns 8 may promote tissue ingrowth into the porous and fibrous core 10 of the palatal implant 2. This scar tissue growth within the interior of the palatal implant 2 may increase the stiffness of the soft palate.

A method of forming a palatal implant 2 may include air jet texturing the triaxial yarns 12. The triaxial yarns 12 may be air jet textured before they are loaded onto the braiding machine 28. When the air jet texturing the triaxial yarns 12, less than 50% of the input yarn may be core yarns and greater than 50% of the input yarns may be effect yarn. In other implementations, greater than 50% of the input yarn may be core yarns and less than 50% of the input yarns may be effect yarn, or 50% of the input yarns may be core yarns and 50% of the input yarns may be effect yarn. When air jet texturing the triaxial yarns 12, the combined over feed of the input yarn may be greater than 20% across both the core yarn and the effect yarn. In other implementations, the combined overfeed of the input yarn may be less than 20% across both the core yarn and the effect yarn or equal to 20% across both the core yarn and effect yarn. In particular implementations, there may be a quantity of one, 100 denier, thirty-six filament core yarn fed into the air jet texturing process at 20% overfeed for every quantity of two, 100 denier, thirty-six filament effect yarn fed into the air jet texturing process at 40% overfeed. The resulting air jet textured triaxial yarns 12 may be a fluffy and low-density fill of yarns. Because the triaxial yarns 12 in the exterior of the palatal implant 2 will be filled with loopy air textured yarns, the exterior of the palatal implant 2 may be highly fibrous. The resulting exterior of the palatal implant 2 may have a low solidity. This low solidity may allow scar tissue to grow throughout the exterior layer of the palatal implant 2 and ultimately into the core 10 of the palatal implant 2. This scar tissue growth within the exterior layer and within the interior of the palatal implant 2 may increase the stiffness of the soft palate.

The biaxial yarns 14 may not be air jet textured. The biaxial yarns 14 may be straight fully drawn yarns. In other implementations, the biaxial yarns 14 may be air jet textured or may be textured in some other manner. The straighter texture of the biaxial yarns 14 in contrast to the “fluffy” texture of the triaxial yarns, may be configured to accentuate the protrusion of the looped filaments 26 of the triaxial yarns 12 between the biaxial yarns 14 when the braided structure 16 is completed. This protrusion of the triaxial yarns 12 through the braided, straight fully drawn biaxial yarns 14, may effectively facilitate the growth of scar tissue growth around and through the triaxial yarns 12 and in turn, throughout the entire palatal implant 2. Importantly, in particular implementations, the braiding of the straight biaxial yarns with the textured triaxial yarns results in a finalized braided structure and palatal implant with a unique linear density of 10,200 denier. While the denier may be less when you add up all the denier of the yarns (core, triaxial, and biaxial), such as 9,280 in particular implementations, because the angle of the biaxial yarns, the denier is increased to weights of 9,800 denier or more, such as 10,200 denier.

The combination of air jet textured core yarns 8, air jet textured triaxial yarns 12, and straight fully drawn biaxial yarns 14 creates a fibrous finalized braided structure 16. The loopy/fibrous finish of the triaxial yarns 12 protrudes through the openings 30 or gaps in the biaxial yarns 14 when the triaxial yarns 12 and biaxial yarns 14 are braided together. This pattern and the exposed loops of the textured triaxial yarns 12 effectively induce fibrotic response and lead scar tissue ingrowth into the core 10 of the implant 2. The looped pattern in the core yarns 8 also allows scar tissue to grow into the core 10 and throughout the interior of the palatal implant 2. This integration of the palatal implant 2 with scar tissue inside of the soft palate may result in significant stiffening of the palatal implant 2 and in turn, stiffening of the soft palate.

The particular implementations of palatal implants disclosed herein may result in a palatal implant that provides an optimal amount of stiffness to prevent or lessen snoring and obstructive sleep apnea.

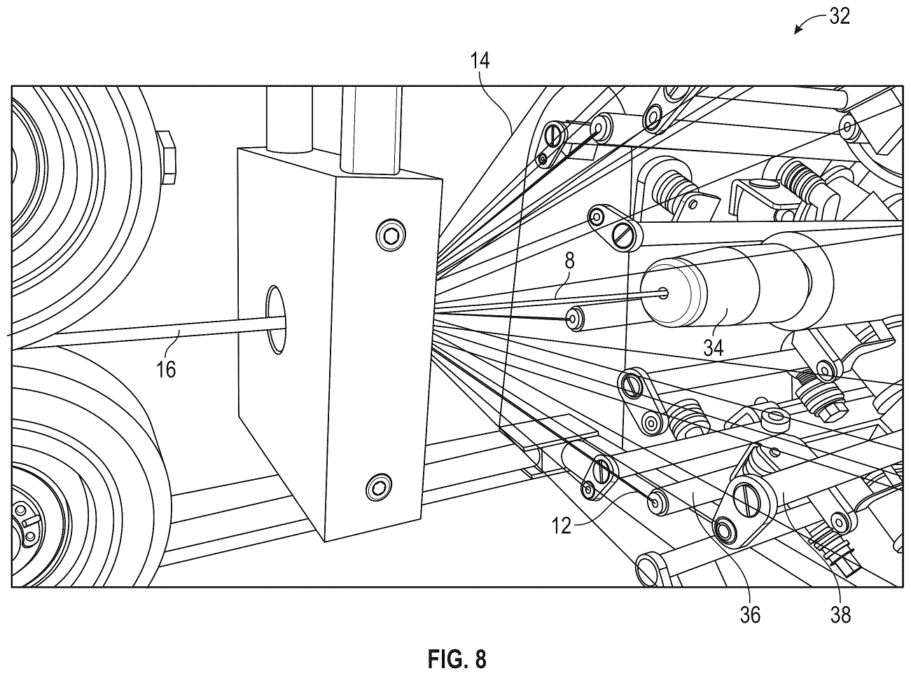

Referring to FIGS. 7-8, a method of constructing a palatal implant 2 may include a braiding machine 32. Referring to FIG. 7, a forty-eight carrier braiding machine 28 is illustrated. Referring to FIG. 8, a loaded braiding machine 32 is illustrated. In particular implementations, the method of forming a palatal implant 2 may include using a forty-eight carrier braiding machine 28 at a half load capacity. In other implementations, the method of forming a palatal implant 2 may include other braiding machines 32 such as, by non-limiting example, a twenty-four carrier braiding machine or a thirty-six carrier braiding machine. When braiding the palatal implant 2, the forty-eight carrier braiding machine 28 may be set to a run speed of 310 rotations per minute (rpm). In other implementations, the forty-eight carrier braiding machine 28 may be set to a run speed of less than 310 rotations per minute or more than 310 rotations per minute. When braiding the palatal implant 2, the pick count of the forty-eight carrier braiding machine 28 may be set to forty-eight picks per inch (ppi). In other implementations, the pick count of the forty-eight carrier braiding machine 28 may be set to between forty picks per inch (ppi) and fifty picks per inch (ppi).

The method of forming the palatal implant may include loading eleven core yarns 8 into core yarn carriers on the center of the braiding machine 32. The core yarn carriers may be located behind the core yarn stems 34 and the carriers may feed the core yarns 8 through the core yarn stems 34. In other implementations, less than eleven core yarns 8 or more than eleven core yarns 8 may be loaded onto the machine. Each individual core yarn 8 carrier may hold one core yarn 8. In other implementations, each core yarn carrier may hold a plurality of core yarns 8. The core yarns 8 may have a total denier weight of 400 denier per yarn. In other implementations, the core yarns 8 may have a weight of 250 denier to 600 denier. The total weight of the core 10 of the palatal implant 2 may be 4400 denier. In other implementations, the total weight of the core 10 of the palatal implant 2 may be less than 4400 denier or more than 4400 denier. The tension of the core yarns 8 may be set to 27 gram-force (gf) per core yarn carrier. In other implementations, the tension of the core yarns 8 may be set to less than 27 gram-force (gf) per core yarn carrier or to more than 27 gram-force (gf) per core yarn carrier.

When loading yarns onto the forty-eight carrier braiding machine 28, eight total triaxial yarns 12 may be loaded into triaxial yarn carriers evenly spaced along the perimeter of the braiding machine 32. The triaxial yarn carriers may be located behind the triaxial yarn stems 36 and carriers may feed the triaxial yarns 12 through the triaxial yarn stems 36. In other implementations, less than eight triaxial yarns 12 or more than eight triaxial yarns 12 may be loaded into triaxial yarn carriers on the braiding machine 32. Each individual triaxial yarn carrier may hold one triaxial yarn 12. The triaxial yarns 12 may have a total denier weight of 400 denier per yarn. In other implementations, the triaxial yarns 12 may have a weight of 250 denier per yarn to 600 denier per yarn.

The method of forming the palatal implant may include loading twenty-four triaxial yarns onto the triaxial carriers of the forty-eight carrier braiding machine. The triaxial yarn carriers that are used may be evenly spaced around the forty-eight carrier braiding machine 28 so that there are two empty triaxial yarn carriers between each used triaxial yarn carrier. In other implementations, the eight triaxial yarns 12 may be spaced unevenly around the forty-eight carrier braiding machine 28. When the eight triaxial yarns 12 are loaded onto the forty-eight carrier braiding machine 28, there may be sixteen empty triaxial yarn carriers. The tension of the triaxial yarns 12 may be set to 78 gram-force (gf) per triaxial yarn carrier. In other implementations, the tension of the triaxial yarns 12 may be set to less than 78 gram-force (gf) per triaxial yarn carrier 36 or to more than 78 gram-force (gf) per triaxial yarn carrier.

In particular implementations, when loading yarns onto the forty-eight carrier braiding machine 28, a quantity of twenty-four total biaxial yarns 14 may be loaded into biaxial yarn carriers 38 at the perimeter of the forty-eight carrier braiding machine 28. In other implementations, less than twenty-four biaxial yarns 14 or more than twenty-four biaxial yarns 14 may be loaded into biaxial yarn carriers 38 on the forty-eight carrier braiding machine 28. Each individual biaxial yarn carrier 38 may hold one biaxial yarn 14. In other implementations, each biaxial yarn carrier 38 may hold more than one biaxial yarn 14. The biaxial yarns 14 may have a total weight of 70 denier per yarn. In other implementations, the biaxial yarns 14 may have a weight of 50 denier per yarn to 100 denier per yarn. The forty-eight carrier braiding machine 28 has forty-eight rotating biaxial yarn carriers 38. The biaxial yarn carriers 38 that are used may be evenly spaced around the forty-eight carrier braiding machine 28 so that there is one empty biaxial yarn carrier 38 between each used biaxial yarn carrier 38. This particular spacing results in the biaxial yarns 14 being arranged in a half load configuration. When twenty-four biaxial yarns 14 are loaded onto the forty-eight carrier braiding machine 28, there may be twenty-four empty biaxial yarn carriers 38.

In particular implementations, the tension of the biaxial yarns 14 may be set to 78 gram-force (gf) per carrier. In other implementations, the tension of the biaxial yarns 14 may be set to less than 78 gram-force (gf) per biaxial yarn carrier 38 or to more than 78 gram-force (gf) per biaxial yarn carrier 38.

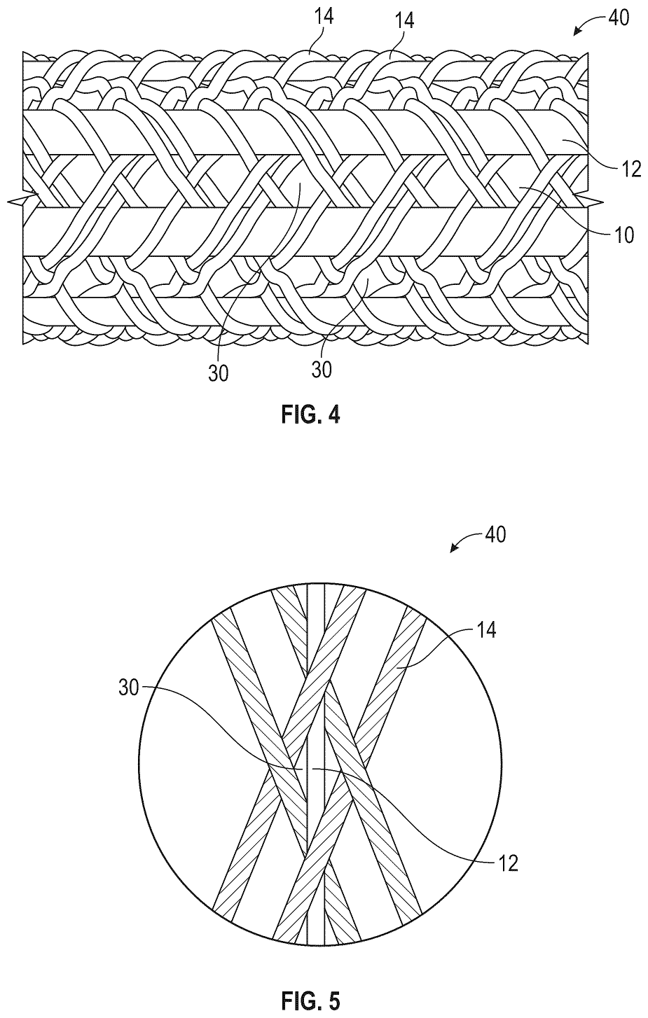

Referring to FIGS. 4-5, a method of forming a palatal implant 2 may include braiding the biaxial yarns 14 and triaxial yarns 12. Referring to FIGS. 4-5, a biaxial yarn 14 and triaxial yarn 12 braiding pattern is illustrated. When all of the yarns are braided together, the core yarns 8 may be oriented horizontally (substantially zero degrees), the triaxial yarns 12 may be oriented horizontally (substantially zero degrees), and the biaxial yarns 14 may be oriented at 45 degrees or negative 45 degrees relative to an axis perpendicular to the length of the palatal implant. In other implementations, the biaxial yarns 14 may be oriented at between 40 degrees and 60 degrees or between negative 40 degrees and negative 60 degrees. The biaxial yarns 14 may be braided together with other biaxial yarns 14 in a 1:1:1 braiding pattem 40, which is also known as a one over one, under one braid. In this 1:1:1 braiding pattern 40, one biaxial yarn 14 from a single biaxial yarn carrier 38 may be woven over the first biaxial yarn 14 that it intersects and then under the next biaxial yarn 14 that it intersects. The one over one under one braiding pattern 40 may continue for every biaxial yarn 14 that the initial biaxial yarn 14 passes. In other implementations, the biaxial yarns 14 may be braided together with other biaxial yarns 14 in, by non-limiting example, a 1:2:2 pattern or 2:2:2 pattern. Simultaneously, the biaxial yarns 14 may be braided together with the triaxial yarns 12 in a 1:1:1 braiding pattern 40. One biaxial yarn 14 from a single biaxial yarn carrier 38 may be woven over the first triaxial yarn 12 that it intersects and then under the next triaxial yarn 12 that it intersects. The over one under one braiding pattern 40 may continue for every triaxial yarn 12 that the biaxial yarn 14 passes. In other implementations, the biaxial yarns 14 may be braided together with the triaxial yarns 12 in a, by non-limiting example, 1:2:2 pattern or 2:2:2 pattern.

When the palatal implant 2 has been successfully braided, the braided structure 16 may be removed from the forty-eight carrier braiding machine 28 and cut to lengths of 240 millimeters (mm). In other implementations, the braided structure 16 may be cut to lengths that are less than 240 millimeters (mm) or more than 240 millimeters (mm). The ends of the 240 millimeter (mm) cut braids may then be fused to prevent unraveling. The ends may be fused by using a torch or some other suitable heat source. The braid may then be washed by placing the cut, 240 millimeter (mm) braid in an ultrasonic bath of pure deionized water for approximately 10 minutes. The ultrasonic bath may remove the spin finish from the braid. The braid may then be dried by being placed in a dryer or oven set to approximately 140 degrees Fahrenheit for approximately 10 minutes.

After being cleaned, the length of the 240 mm cut braid may be loaded into an ultrasonic welding fixture. The welding may cause the braid to contract. One of the fused, hardened ends of the braid may be positioned just beyond the side of the ultrasonic welding fixture to anchor the braid and constrain braid shrinkage to a single direction. The fixture may then be placed into the ultrasonic welding setup with the anchored side of the braid underneath the welding head. The fixture may be positioned so that the indexer is aligned with the first weld location. The welding head may be pressed downwards until it makes contact with the braid. While continuing to push down into the braid, the welder may be activated until the weld is complete. When the weld is complete, the welder may turn off automatically. When the weld is complete, the downward pressure may slowly be released to allow the welder to lift away from the braid. The fixture may then be moved so that it is indexed on the second weld 20 location. The welding steps may be repeated until all 12 implants have been welded on both ends. The braid may remain in the channel along the entire length of the fixture and ensure that the anchored end remains fixed at the end of the fixture.

The anchor end of the braid may then be pierced downward onto a needle fixture that may be secured in a desktop vice. The needle may be threaded with PET thread or a medical suture using a needle threader. The braid may be removed from the needle and one end of the thread may be pulled all the way through the braid. Both ends of the thread may be passed through the loading funnel and subsequently may pass through a piece of implant sheathing. Passing through the sheathing may require a specialized sheathing tool. The hard, fused end of the braid may be cut and pulled into the funnel at an angle, forming a spear shape to facilitate loading into the funnel. The end of the braid may be guided into the loading funnel by holding both the funnel and the sheath together in one hand and pulling on the polyester thread with another hand to pull the braid into the sheathing until the leading end of the braid meets the far end of the sheath. The polyester thread may be removed from the braid and the sheath (now loaded with the braid) may be removed from the loading funnel. The sheathed braid may then be cut into 12 individual palatal implants 2 through the following guidelines. The cut may land 0.8 millimeters to 1.2 millimeters from the outside of the first weld 18 location. The same cutter may be used in combination with a length guide and the sheathed braid may then be cut at 18 mm increments, producing 12 cut palatal implants 2 from the length of the braid. Finally, the palatal implants 2 may be kept in sheaths for full device assembly while the excess ends of the braid may be discarded.

The palatal implant 2 may be configured to be implanted in a patient's soft palate by a medical delivery tool which houses a hypodermic needle. In other implementations, the palatal implant 2 may be configured to be implanted in a patient's soft palate by any other medical implantation device or process. The palatal implant 2 may be implanted in the soft palate of a patient that is suffering from snoring, or obstructive sleep apnea. The palatal implant 2 may be implanted through a standard 14RW gauge hypodermic needle that can enter the soft palate under local anesthesia. The hypodermic needle may radially pre-compress the palatal implant 2 and prevent premature splaying of the first end 22 and the second end 24 of the palatal implant 2. A tool may guide the sheathed palatal implant 2 into the back end or funnel side of the needle. A plunge wire may then be used to push the palatal implant 2 out of the sheathing and into the needle. The length of the palatal implant 2 may be optimized to fit the length of the average person's palate length. In other implementations, the palatal implant 2 size may be altered throughout the method of forming a palatal implant 2. This alteration may allow the palatal implant 2 to fit inside the soft palate of a patient that does not have an average sized soft palate.

In places where the description above refers to particular implementations of palatal implants and implementing components, sub-components, methods and sub-methods, it should be readily apparent that a number of modifications may be made without departing from the spirit thereof and that these implementations, implementing components, sub-components, methods and sub-methods may be applied to other palatal implants.

What is claimed is:

1. A palatal implant comprising:
a core comprising a plurality of air jet textured core yarns;
8 triaxial yarns coupled around the core and parallel to the core, wherein each of the 8 triaxial yarns are air jet textured and comprise a plurality of looped filaments;
24 biaxial yarns braided around the core in a one over one under one pattern;
a first weld offset from a first end of the palatal implant by 0.8 millimeters to 1.2 millimeters; and
a second weld offset from a second end of the palatal implant by 0.8 millimeters to 1.2 millimeters;
wherein the looped filaments in the triaxial yarns protrude through openings between the biaxial yarns;
wherein the triaxial yarns each comprise a weight of between 250-600 denier;
wherein the biaxial yarns each comprises a weight of between 50-100 denier;
wherein the palatal implant has a flexural modulus between 1.0 MPa and 3.0 MPa;
wherein the palatal implant comprises a total braid weight of 7,000 denier to 12,500 denier; and
wherein the palatal implant is configured to stiffen a palate through tissue ingrowth into the palatal implant.

2. The palatal implant of claim 1, wherein the core yarns, the biaxial yarns, and the triaxial yarns are comprised of polyethylene terephthalate (PET).

3. The palatal implant of claim 1, wherein the palatal implant comprises a length of less than 20 millimeters.

4. The palatal implant of claim 1, wherein the palatal implant comprises a diameter between 1 millimeter and 2 millimeters.

5. The palatal implant of claim 1, wherein the palatal implant comprises a linear density of 0.78 grams per meter (g/m) to 1.39 grams per meter (g/m).

6. The palatal implant of claim 1, wherein a sum of denier of all yarns in the palatal implant is substantially 9280 and a total braid weight of the palatal implant is 9800 or more.

7. The palatal implant of claim 1, wherein each of the 24 biaxial yarns are flat drawn yarns.

8. The palatal implant of claim 1, wherein each of the plurality of air jet textured core yarns is 400 denier and contains 108 filaments per yarn, each of the 8 triaxial yarns is 400 denier and contains 108 filaments per yarn, and each of the 24 biaxial yarns is 70 denier and contains 30 filaments per yarn.

9. The palatal implant of claim 1, wherein the first end of the palatal implant and the second end of the palatal implant are configured to fray and bond with scar tissue when implanted into the palate.

10. A method of constructing a palatal implant comprising:
air jet texturing a plurality of core yarns;
air jet texturing 8 triaxial yarns;
loading and evenly spacing the 8 triaxial yarns on 8 carriers of a 48 carrier braiding machine;
loading 24 biaxial yarns on 24 carriers of the 48 carrier braiding machine, each of the 24 biaxial yarns oriented one of between 40 degrees and 60 degrees or between negative 40 degrees and negative 60 degrees;
braiding the 24 biaxial yarns in a one under one over one configuration to form a braided structure; and
forming a plurality of welds along the braided structure;
wherein the 48 carrier braiding machine is loaded in a half-load configuration when the 8 triaxial yarns and the 24 biaxial yarns are loaded onto the 48 carrier braiding machine.

11. The method of claim 10, wherein the air jet texturing of the core yarns and the air jet texturing of the triaxial yarns comprises less than 50% of input yarn as core yarn and greater than 50% of the of the input yarn as effect yarn.

12. The method of claim 10, wherein the braided structure of the palatal implant comprises a total weight of 7,000 denier to 12,500 denier.

13. The method of claim 10, wherein a mean height of a surface of a texture of the palatal implant is greater than 50 micrometers.

14. The method of claim 11, wherein the combined over feed of the input yarn is greater than 20% across both the core yarn and the effect yarn.

15. The method of claim 10, wherein each of the plurality of air jet textured core yarns is 400 denier and contains 108 filaments per yarn, each of the 8 triaxial yarns is 400 denier and contains 108 filaments per yarn, and each of the 24 biaxial yarns is 70 denier and contains 30 filaments per yarn.

16. A method of constructing a palatal implant comprising:
air jet texturing a plurality of core yarns;
air jet texturing 8 triaxial yarns;
loading and evenly spacing the 8 triaxial yarns on 8 carriers of a 48 carrier braiding machine;
loading 24 biaxial yarns on 24 carriers of the 48 carrier braiding machine, each of the 24 biaxial yarns oriented one of between 40 degrees and 60 degrees or between negative 40 degrees and negative 60 degrees;
braiding the 24 biaxial yarns in a one under one over one configuration to form a braided structure;
forming a plurality of welds along the braided structure; and
cutting the braided structure to form a plurality of palatal implants;
wherein the 48 carrier braiding machine is loaded in a half-load configuration when the 8 triaxial yarns and the 24 biaxial yarns are loaded onto the 48 carrier braiding machine;
wherein the triaxial yarns of the plurality of palatal implants comprise looped filaments that protrude through openings between the biaxial yarns;
wherein the triaxial yarns of the plurality of palatal implants each comprise a weight of between 250-600 denier;
wherein the biaxial yarns of the plurality of palatal implants each comprises a weight of between 50-100 denier;
wherein the palatal implant has a flexural modulus between 1.0 MPa and 3.0 MPa;
wherein the palatal implant comprises a total denier weight of 7,000 denier to 12,500 denier; and
wherein the palatal implant is configured to stiffen a palate through tissue ingrowth into the palatal implant.

17. The method of claim 16, wherein the plurality of palatal implants comprise a pick count of 40 picks per inch (ppi) to 50 picks per inch (ppi).

18. The method of claim 16, wherein the air jet texturing of the core yarns and the air jet texturing of the triaxial yarns comprises less than 50% of input yarn as core yarn and greater than 50% of the input yarn as effect yarn.

19. The method of claim 16, wherein the braided structure of the palatal implant comprises a linear density of 0.78 grams per meter (g/m) to 1.39 grams per meter (g/m).

20. The method of claim 16, wherein each palatal implant of the plurality of palatal implants comprises a first weld between 0.8-1.2 mm from a first end and a second weld between 0.8-1.2 mm from a second end.
